(19) United States
(12) Patent Application Publication
(10) Pub. No.: US 2007/0065379 A1
(43) Pub. Date: Mar. 22, 2007

(54) Microcapsules having an aqueous core and their use in cosmetics

(75) Inventors: Bruno Biatry, Vincennes (FR); Jean-Thierry Simonnet, Cachan (FR); Eric Lheureux, Montgeron (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/520,699

(22) Filed: Sep. 14, 2006

Related U.S. Application Data
(60) Provisional application No. 60/718,296, filed on Sep. 20, 2005.

(30) Foreign Application Priority Data
Sep. 15, 2005 (FR) 05 52782

(51) Int. Cl. A61K 8/73; B01J 13/04

(52) U.S. Cl. 424/59; 424/70.17; 264/4.1

(57) ABSTRACT

Microcapsules containing a polymeric and/or waxy envelope encapsulating an aqueous medium containing an acrylic polymer containing a phosphorylcholine group. Composition containing these microcapsules. Application to, caring for, and to making up keratinous substances.

### REFERENCE TO PRIOR APPLICATIONS

This application claims priority to U.S. provisional application 60/718,296 filed Sep. 20, 2005, and to French patent application 0552782 filed Sep. 15, 2005, both incorporated herein by reference.

### FIELD OF THE INVENTION

The present invention relates to microcapsules comprising an aqueous core comprising at least one moisturizing polymer comprising a phosphorylcholine group, to cosmetic or dermatological compositions such as cosmetic or dermatological compositions comprising them and to a process for preparing such microcapsules, among other things.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The microencapsulation of cosmetic or dermatological active principles for the purpose of better preservation and/or of prolonged and controlled release is known. Microencapsulation processes and the principles to which they resort are described in detail, for example, in “Microencapsulation, Methods and Industrial Application”, edited under the direction of Benita, M. Dekker, 1996. In these processes, the wall of the microparticle is formed from polymers which will isolate the active principle from the external medium. These microcapsules make it possible to encapsulate higher levels of water-soluble active principles than other vesicular systems, such as liposomes, which are nanoparticles formed of double phospholipid layers surrounding an aqueous core.

Application EP-A-1 151 741 discloses microcapsules comprising an aqueous core which are formed of a polymeric and/or waxy envelope encapsulating an active principle. These microcapsules are obtained by a multiple emulsification/solvent evaporation preparation process. This process comprises, as first stage, the preparation of a water-in-oil primary emulsion obtained by dispersion of an aqueous composition comprising the active principle in an organic solution of the polymer and/or of the wax forming the envelope of the microcapsule and then, as second stage, the dispersion of the primary emulsion in an external aqueous phase. The organic solvent is subsequently removed by evaporation.

The active principle used during the preparation of the microcapsules has an effect on the quality of the primary emulsion.

The encapsulated active principle can also modify the microcapsules over time. Thus, when the active principle has a low molecular weight, for example of less than 100 g/mol, such as glycerol, it plasticizes the polymer forming the wall of the microcapsules; the microcapsules swell over time and end up bursting. The microcapsules are thus not stable and it is thus not possible to encapsulate glycerol.

Furthermore, some polymer active principles, such as hyaluronic acid or DNA, or also some thickening polymers, such as guar gum, form aqueous solutions of high viscosity at relatively low contents (of the order of 1% by weight in water). In point of fact, these aqueous solutions of high viscosity are harmful to the preparation of the primary emulsion as it is difficult, indeed even impossible, to disperse the aqueous phase of the active principle or of the additive in the organic solvent. The emulsion possibly obtained exhibits excessively large droplets which do not make it possible to form microcapsules. These excessively viscous active principles can only be encapsulated at very low contents which then do not make it possible to obtain microcapsules having good effectiveness of the active principle.

It is thus not obvious to a person skilled in the art to be able to satisfactorily obtain microcapsules encapsulating an active principle in an aqueous medium, in particular with a high content of active principle.

### OBJECTS OF THE INVENTION

One object of the present invention is to make available microcapsules comprising an aqueous core encapsulating a moisturizing active principle, in particular employing a large amount of moisturizing active principle, thus making it possible to obtain improved moisturizing properties, in particular with compositions comprising these microcapsules.

### SUMMARY OF THE INVENTION

The inventors have discovered, surprisingly, microcapsules comprising an aqueous core exhibiting improved moisturizing properties using, as encapsulated active principle, an acrylic polymer having a group of phosphorylcholine type.

### DETAILED DESCRIPTION OF THE PREFERRED EMBODIMENTS

Aqueous compositions comprising an acrylic polymer having a phosphorylcholine group, in particular moisturizing cosmetic compositions for caring for the skin, are known from the documents US-A-5 468 475, EP-A-767 212 and EP-A-1 163 905. However, these documents do not disclose microcapsules comprising an aqueous core comprising the acrylic polymer; neither do they suggest that such an acrylic polymer can be encapsulated in microcapsules.

The acrylic polymer having a phosphorylcholine group is readily soluble or dispersible in water and makes it possible to obtain an aqueous solution not exhibiting a high viscosity, even at high contents of the acrylic polymer, in particular at contents of greater than 1% by weight, in particular which can range up to 40% by weight. It is thus possible to encapsulate the acrylic polymer in high amounts and to thus obtain microcapsules exhibiting a good moisturizing activity.

Furthermore, the encapsulation of the acrylic polymer makes it possible to improve the cosmetic properties of compositions comprising these microcapsules in comparison with a composition comprising the polymer nonencapsulated: the composition applied to the skin is more moisturizing and less tacky.

A subject-matter of the present invention is thus microcapsules comprising a polymeric and/or waxy envelope encapsulating an aqueous medium comprising an acrylic polymer comprising a phosphorylcholine group.

Another subject-matter of invention is a process for the manufacture of microcapsules comprising an aqueous core and comprising a polymeric and/or waxy envelope encapsulating an aqueous medium comprising an acrylic polymer comprising a phosphorylcholine group which are described above by multiple emulsification/solvent evaporation.

Another subject-matter of the invention is a composition, in particular a cosmetic or dermatological composition, comprising, in a physiologically acceptable medium, the microcapsules comprising an aqueous core and comprising a polymeric and/or waxy envelope encapsulating an aqueous medium comprising an acrylic polymer comprising a phosphorylcholine group.

A further subject-matter of the invention is a non-therapeutic method for caring for or making up keratinous substances comprising the application, to the keratinous substances, of a composition as described above.

The microcapsules according to the invention comprise an encapsulated aqueous medium comprising an acrylic polymer comprising a phosphorylcholine group, referred to in the continuation of this description as a PC acrylic polymer.

The PC acrylic polymer is advantageously a water-soluble or water-dispersible polymer and preferably a water-soluble polymer.

The term “water-soluble polymer” is understood to mean a polymer having a solubility of at least 0.1% by weight in water at 25° C. The term “water-dispersible polymer” is understood to mean a polymer capable of being dispersed homogeneously in water at 25° C. without forming a nonhomogeneous phase.

The term “acrylic polymer comprising a phosphorylcholine group” is understood to mean a polymer having an acrylic backbone and comprising pendent groups (or side chains) comprising at least one group of following formula (I): 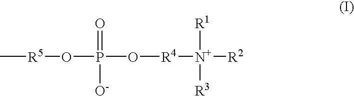
in which R¹, R² and R³ independently denote an alkyl group having from 1 to 8 carbon atoms; R⁴ denotes —(CH₂—CHR₆O)ₘ—(CH₂—CHR₆)ₚ— with R₆ denoting a hydrogen atom or a methyl or ethyl group and m denoting an integer ranging from 0 to 10 and p denoting an integer ranging from 1 to 2; R⁵ denotes —(CH₂)_{g}—, g being an integer ranging from 2 to 10.

Such a polymer can be obtained by polymerization of an acrylic monomer comprising the group of formula (I) described above, referred to in the continuation of this description as PC acrylic monomer.

Advantageously, the PC acrylic monomer is a monomer corresponding to the following formula (II): 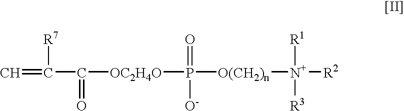
in which R¹, R² and R³ independently denote an alkyl group having from 1 to 8 carbon atoms; n representing an integer ranging from 2 to 4; R⁷ denotes a hydrogen atom or a methyl group.

Mention may be made, as preferred PC acrylic monomers, of the following monomers:

2-(meth)acryloyloxyethyl 2′-(trimethylammonio)ethyl phosphate, 3-(meth)acryloyloxypropyl 2′-(trimethylammonio)ethyl phosphate, 4-(meth)acryloyloxybutyl 2′-(trimethylammonio)ethyl phosphate, 5-(meth)acryloyloxypentyl 2′-(trimethylammonio)ethyl phosphate, 2-(meth)acryloyloxyethyl 2′-(triethylammonio)ethyl phosphate, 3-(meth)acryloyloxypropyl 2′-(triethylammonio)ethyl phosphate, 4-(meth)acryloyloxybutyl 2′-(triethylammonio)ethyl phosphate, 5-(meth)acryloyloxypentyl 2′-(triethylammonio)ethyl phosphate, 2-(meth)acryloyloxyethyl 2′-(tripropylammonio)ethyl phosphate, 3-(meth)acryloyloxypropyl 2′-(tripropylammonio)ethyl phosphate, 4-(meth)acryloyloxybutyl 2′-(tripropylammonio)ethyl phosphate, 5-(meth)acryloyloxypentyl 2′-(tripropylammonio)ethyl phosphate, 2-(meth)acryloyloxyethyl 2′-(tributylammonio)ethyl phosphate, 3-(meth)acryloyloxypropyl 2′-(tributylammonio)ethyl phosphate, 4-(meth)acryloyloxybutyl 2′-(tripropylammonio)ethyl phosphate, 5-(meth)acryloyloxypentyl 2′-(tributylammonio)ethyl phosphate, 2-(meth)acryloyloxyethyl 3′-(trimethylammonio)propyl phosphate, 2-(meth)acryloyloxyethyl 3′-(triethylammonio)propyl phosphate, 2-(meth)acryloyloxyethyl 4′-(triethylammonio)butyl phosphate, 2-(meth)acryloyloxyethyl 3′-(tripropylammonio)propyl phosphate, 2-(meth)acryloyloxyethyl 4′-(tripropylammonio)butyl phosphate, 2-(meth)acryloyloxyethyl 3′-(tributylammonio)propyl phosphate, 2-(meth)acryloyloxyethyl 4′-(tributylammonio)butyl phosphate, 3-(meth)acryloyloxyethyl 3′-(trimethylammonio)propyl phosphate, 3-(meth)acryloyloxypropyl 4′-(trimethylammonio)butyl phosphate, 3-(meth)acryloyloxypropyl 3′-(triethylammonio)propyl phosphate, 3-(meth)acryloyloxypropyl 4′-(triethylammonio)butyl phosphate, 3-(meth)acryloyloxypropyl 3′-(tripropylammonio)propyl phosphate, 3-(meth)acryloyloxypropyl 4′-(tripropylammonio)butyl phosphate, 3-(meth)acryloyloxypropyl 3′-(tributylammonio)propyl phosphate, 3-(meth)acryloyloxypropyl 4′-(tributylammonio)butyl phosphate, 4-(meth)acryloyloxybutyl 3′-(trimethylammonio)propyl phosphate, 4-(meth)acryloyloxybutyl 4′-(trimethylammonio)butyl phosphate, 4-(meth)acryloyloxybutyl 4′-(triethylammonio)butyl phosphate, 4-(meth)acryloyloxybutyl 3′-(tripropylammonio)propyl phosphate, 4-(meth)acryloyloxybutyl 4′-(tripropylammonio)butyl phosphate, 4-(meth) acryloyloxybutyl 3′-(tributylammonio)propyl phosphate, and 4-(meth)acryloyloxybutyl 4′-(tributylammonio)butyl phosphate,

Mixtures may be used.

Use is preferably made, as PC acrylic monomer, of 2-(meth)acryloyloxyethyl 2′-(trimethylammonio)ethyl phosphate, also referred to as 2-(methacryloyloxyethyl)-phosphorylcholine.

Preferably, the PC acrylic polymer used according to the invention is a polymer obtained by polymerization of at least one PC acrylic monomer as described above and optionally of one or more additional monomers other than the at least one PC acrylic monomer.

The additional monomers can be chosen for example from methyl (meth)acrylate, ethyl (meth)acrylate, butyl (meth)acrylate, (meth)acrylic acid, (meth)acrylamide, 2-hydroxyethyl (meth)acrylate, ethyl vinyl ether, butyl vinyl ether, N-vinylpyrrolidone, vinyl chloride, ethylene, isobutylene, acrylonitrile, styrene, methylstyrene or (chloromethyl)styrene.

Preferably, the PC acrylic polymer can comprise from 40 to 100 mol % of units resulting from the one or more PC acrylic monomers as described above and from 0 to 60 mol % of units resulting from additional monomer.

Preferably, the polymer comprising a phosphorylcholine group is chosen from 2-(methacryloyloxyethyl)phosphorylcholine homopolymer, 2-(methacryloyloxyethyl)phosphorylcholine/butyl methacrylate copolymer, 2-(methacryloyloxyethyl)phosphorylcholine/2-hydroxy-3-methacryloyloxypropyltrimethylammonium chloride copolymer, 2-(methacryloyloxyethyl)phosphoryl-choline/butyl methacrylate/sodium methacrylate terpolymer or 2-(methacryloyloxyethyl)phosphoryl-choline/stearyl methacrylate copolymer.

Preferably, use is made of 2-(methacryloyloxyethyl)-phosphorylcholine homopolymer or 2-(methacryloyl-oxyethyl)phosphorylcholine/butyl methacrylate copolymer and more preferably of 2-(methacryloyloxyethyl)-phosphorylcholine homopolymer.

Such polymers are disclosed in the documents EP-A-1 163 905, EP-A-1 095 665, FR-A-2 698 003 and EP-A-767 212, the contents of which are incorporated by way of reference in the present application.

The PC acrylic polymer preferably has a weight-average molecular weight ranging from 50 000 to 1 000 000 and preferably ranging from 80 000 to 800 000.

Use may be made, as acrylic polymer comprising a phosphorylcholine group in accordance with the invention, of one or more of:
the poly[2-(methacryloyloxyethyl)phosphorylcholine] at 40% in a water/butanediol mixture (5% of butanediol) sold under the name Lipidure HM by Nippon Oils and Fats; this product has the CTFA name: Polyphosphorylcholine glycol acrylate (and) butylene glycol.
the 2-(methacryloyloxyethyl)phosphorylcholine/butyl methacrylate (90/10) copolymer at 5% in solution in water sold under the name Lipidure PMB by Nippon Oils and Fats; this product has the CTFA name: Polyquaternium-51,
the 2-(methacryloyloxyethyl)phosphorylcholine/2-hydroxy-3-methacryloyloxypropyltrimethylammonium chloride copolymer at 5% in solution and water sold under the name Lipidure C by Nippon Oils and Fats,
the 2-(methacryloyloxyethyl)phosphorylcholine/butyl methacrylate/sodium methacrylate terpolymer at 5% in solution and water sold under the name Lipidure-A by Nippon Oils and Fats,
the 2-(methacryloyloxyethyl)phosphorylcholine/stearyl methacrylate copolymers sold under the names Lipidure-S, Lipidure-NR and Lipidure-NA by Nippon Oils and Fats; these products have the CTFA name: Polyquaternium-61.

The PC acrylic polymer can be present in the aqueous medium encapsulated in the microcapsules in any amount but preferably in a content ranging from 0.1% to 40% by weight, with respect to the total weight of the encapsulated aqueous phase, preferably ranging from 1% to 40% by weight, more preferably ranging from 2% to 40% by weight, preferably ranging from 5% to 40% by weight, preferentially ranging from 5% to 30% by weight, and more preferentially ranging from 10% to 25% by weight, including all values and subranges between all stated ranges, and of course including endpoints thereof.

The envelope of the microcapsules (or wall) can comprise at least one water-insoluble polymer and/or one wax.

The term “water-insoluble polymer” is understood to mean a polymer having a solubility in water at 25° C. of less than 0.1% by weight.

The water-insoluble polymer of the envelope can for example be chosen from:
polycaprolactone (such as that sold under the name CAPA640 by Solvay), polybutyrolactone or poly(3-hydroxybutyrate);
poly(C₂-C₆ alkylene adipate)s, such as poly(ethylene adipate) or poly(butylene adipate).

The term “poly(alkylene adipate)” encompasses both homopolymers of adipic acid and of an alkanediol and copolymers of poly(esterether) type, which are linear or branched, obtained from adipic acid and from one or more alkanediols and/or ether diols and/or triols.

The alkanediols used for the preparation of the poly(alkylene adipate)s include linear- or branched-chain C₂₋₆ alkanediols chosen from ethylene glycol, propylene glycol, 1,3-propanediol, 1,4-butanediol, 1,5-pentanediol, 1,6-hexanediol and neopentyl glycol. The ether diols are di-, tri- or tetra(C₂₋₄ alkylene) glycols, such as diethylene glycol, triethylene glycol, tetraethylene glycol, dipropylene glycol, tripropylene glycol, tetrapropylene glycol or dibutylene glycol, tributylene glycol or tetrabutylene glycol.

As indicated above, the polyesters of poly(alkylene adipate) type used for the preparation of the microcapsules of the invention can also comprise a limited number of branching units derived from triols.

The triols used are generally chosen from glycerol, trimethylolethane and trimethylolpropane.

Poly(alkylene adipate) polymers are disclosed in particular in Application EP-A-1 029 587.
polyester polyols of adipic acid and of butanediol, such as polyester of adipic acid, of 1,4-butanediol and of 2-ethyl-2-(hydroxymethyl)-1,3-propanediol (such as Lexorez® 1151-35 from Inolex). Polyester polyols of adipic acid and butanediol are disclosed in particular in the application EP-A-1 342 471.
esters of cellulose and of at least one C₁-C₄ carboxylic acid, such as cellulose acetate, cellulose acetate/propionate (for example those sold under the names “CAP-482-0.5”, “CAP-482-20” and “CAP-504” by Eastman Chemical), cellulose acetate/butyrate (for example those sold under the names “CAB-551”, “CAB-500”, “CAB 553” and “CAB-381” by Eastman Chemical), preferably from cellulose acetate/butyrate and cellulose acetate/propionate;
poly(ortho ester)s obtained by polycondensation of polyol, of diol lactide and of 3,9-diethylidene-2,4,8,10-tetraoxaspiro[5.5]undecane, such as those disclosed in the publication Bouchemal K., Microencapsulation of dehydroepiandrosterone (DHEA) with poly(ortho ester) polymers by interfacial polycondensation, Journal of microencapsulation, 2003, vol. 20, No. 5, 637-651;
poly(ethylene glycol)-poly(butylene terephthalate) block polymers, such as those disclosed in the publication Bezemer J. M., Microspheres for protein delivery prepared from amphiphilic multiblock copolymers, Journal of Controlled Release, 67 (2000), 233-248;
poly(ethylene glycol terephthalate/polybutylene terephthalate) copolymers, such as those disclosed in U.S. Pat. No. 5,980,948;
copolymers of styrene and of maleic anhydride, such as those sold under the name SMA by Cray Valley;
copolymers of styrene and of acrylic acid, such as that sold under the name Plioway Ultra 200 by Plioway;
styrene-ethylene/butylene-styrene block terpolymers and styrene-ethylene/propylene-styrene block terpolymers, such as those sold under the name Kraton G by Shell;
terpolymers of ethylene, of vinyl acetate and of maleic anhydride, such as those sold under the name Arevac by Arkema;
and their mixtures.

The wax of the envelope can be chosen for example from beeswax, polyglycerolated beeswax, hydrogenated vegetable oils, paraffin wax with a melting point of greater than 45° C., and silicone waxes.

Mention may be made, as silicone waxes, for example, of alkyl or alkoxy dimethicones comprising from 16 to 45 carbon atoms, such as behenoxy dimethicone, and C₁₆₋₄₅ alkyl esters of dimethiconol, such as dimethiconol behenate.

A wax, within the meaning of the present invention, is a lipophilic compound which is solid at ambient temperature (approximately 25° C.), which exhibits a reversible solid/liquid change in state, which has a melting point of greater than approximately 40° C. which can range up to 200° C. and which exhibits an anisotropic crystalline arrangement in the solid state.

The microcapsules according to the present invention make it possible to obtain degrees of encapsulation of the PC acrylic polymer of greater than or equal to 25%, indeed even of greater than 60%, including 10, 15, 20, 30, 35, 40, 45, 50, and 55%, with respect to the weight of the active principle employed including all values and subranges between all stated ranges, and of course including endpoints thereof.

The aqueous medium encapsulated in the microcapsules can comprise one or more additional water-soluble polymers other than the polymer comprising a PC group described above.

The additional water-soluble polymers can be chosen in particular from poly(vinyl alcohol), polyvinyl-pyrrolidone, carboxymethylcellulose, poly(carboxylic acid)s and the crosslinked derivatives of these, and natural gums, such as xanthans, starch, sodium alginate, pectins, chitosan, guar, locust bean, carrageenan or hyaluronic acid. These additional water-soluble polymers can preferably be present in a content ranging from 0.01 to 10%, preferably in a proportion of 0.1 to 5%, by weight, with respect to the total weight of the encapsulated aqueous medium.

The encapsulated aqueous medium can comprise one or more salts which can be chosen from sodium chloride, potassium chloride, magnesium sulfate and magnesium chloride. The salt can preferably be present in a content ranging from 0.1 to 10% by weight, with respect to the total weight of the encapsulated aqueous medium, and more preferably ranging from 0.1 to 5% by weight.

The microcapsules can in particular be univacuolar or multivacuolar microcapsules, that is to say the external envelope can include a single aqueous phase compartment or else the internal aqueous phase can be divided into a multitude of compartments separated by walls of the same chemical nature as the external envelope. This phenomenon generally occurs when the multiple emulsion is particularly stable and gives excellent encapsulation results.

The ratio by weight of the encapsulated aqueous medium forming the core of the microcapsules of the present invention to the envelope of the microcapsules (polymer and/or wax) is preferably generally between 0.1/1 and 50/1 and preferably between 0.5/1 and 10.1.

The microcapsules of the present invention generally have a mean diameter of between 1 μm and 1000 μm and more particularly between 1 and 80 μm.

Another subject-matter of the present invention is a process for the manufacture of the microcapsules comprising an aqueous core comprising at least one PC acrylic polymer and a polymeric and/or waxy envelope as are described above. This process is a process for microencapsulation by multiple emulsification/solvent evaporation comprising the following successive stages:
(a) dissolution of at least one PC acrylic polymer in an aqueous medium,
(b) emulsification of the aqueous solution obtained in stage (a) in a solution of at least one water-insoluble polymer and/or of at least one wax in a water-immiscible organic solvent,
(c) emulsification of the water-in-oil primary emulsion obtained in stage (b) in an aqueous solution preferably comprising an agent for stabilizing the emulsion,
(d) removal of the organic solvent by evaporation, giving an aqueous suspension of microcapsules.

The aqueous solution prepared in stage (a) advantageously comprises the PC acrylic polymer in a content similar to those described above for the aqueous medium encapsulated in the microcapsules.

The nature of the water-immiscible organic solvent used in stage (b) is generally chosen according to its solvating power with respect to the material of the wall, to its solubility in water, which has to be as low as possible, and to its boiling point, which is preferably less than 100° C. Use may be made, for example, of dichloromethane, cyclohexane, heptane, 1-chlorobutane, ethyl acetate, ethyl formate or dimethoxymethane (or methylal).

The stability of the multiple emulsion is a determining factor for obtaining good encapsulation results. This is because inadequate stability of the multiple emulsion would result in mixing of the internal and external aqueous phases and escape of the active principle from the vesicles formed. It is consequently highly recommended to add, to the continuous aqueous phase of stage (c), an agent for stabilizing the emulsion.

Appropriate polymeric stabilizing agents are known in the art and can be chosen, for example, from poly(vinyl alcohol), polyvinylpyrrolidone, water-soluble styrene/maleic anhydride copolymers, carboxymethylcellulose, starch, chitosan and polyacrylic acid.

Although the use of the polymeric stabilizing agent is preferable, it is also possible to use, in place of the latter, water-soluble surfactants.

For the purpose of increasing the stability of the multiple emulsion, the continuous aqueous phase of the latter can also comprise from 0.1 to 10% by weight, with respect to the total weight of the continuous aqueous phase, of one or more inorganic salts chosen, for example, from sodium chloride, potassium chloride, magnesium sulfate and magnesium chloride.

It is also possible to introduce a surfactant into the organic solvent solution of stage (b) in order to improve the stability of the primary emulsion. A person skilled in the art will know how to choose the appropriate compound based on this disclosure, such as, for example, surfactants with an HLB (hydrophilic-lipophilic balance) of less than 10, such as esters of sorbitan and of fatty acids, such as, for example, polysorbates, fat-soluble lecithins, monoglycerides of fatty acids, PEG-30 dipolyhydrostearate (Arlacel® P135 from ICI), cetyl dimethicone copolyol (Abil® EM90 from Goldschmidt), oxyethylenated polydimethylsiloxane (DC2-5695® from Dow Chemical), polyolefins comprising a succinic ending (Lubrizol 5603® from Lubrizol, Chemcinnate 1000 AF or 2000 from Chemron) or glycoglycerides, such as those disclosed in EP-A-1 151 745.

The multiple emulsification/solvent evaporation process is already known from U.S. Pat. Nos. 3,523,906 and 3,523,907 and from Application EP-A-1 151 741.

The size and internal structure of the microcapsules depends on a very large number of parameters related to the manufacturing process, such as the temperature, the stirring speed during the emulsification, the chemical nature and the respective amounts of the various water-soluble and organosoluble components, the amount of stabilizing agents, and the like. A person skilled in the art will know how to vary these various parameters in order to obtain the desired microcapsule morphology in view of this disclosure.

The suspension of microcapsules obtained after the evaporation of a solvent can be used as is or can, for example, be incorporated in a cosmetic or dermatological composition.

If desired, the microcapsules can also be separated from the aqueous suspension obtained in stage (d) by filtration and can be dried, so as to obtain a microcapsule powder.

The invention also relates to a composition comprising, in a physiologically acceptable medium, the micro-capsules described herein.

The term “physiologically acceptable medium” is understood to mean a medium compatible with the keratinous substances of human beings. It is preferably a cosmetically or dermatologically acceptable medium, that is to say one which exhibits a pleasing color, a pleasing smell and a pleasing feel and which does not cause unacceptable discomfort (tingling, tightness, redness) liable to dissuade the consumer from using this composition.

The microcapsules (core and wall) can be present in the composition according to the invention in any amount including a content ranging from 0.1% to 30% by weight, with respect to the total weight of the composition, and preferably ranging from 0.1% to 10% by weight.

The PC acrylic polymer can be present in the composition according to the invention in any amount but preferably in a content ranging from 0.01% to 10% by weight, with respect to the total weight of the composition, and more preferably ranging from 0.01% to 5% by weight.

The composition according to the invention can comprise water and/or an organic solvent which is miscible with water at 25° C. and/or an oil.

The composition according to the invention can comprise an organic solvent which is miscible with water at 25° C. chosen in particular from lower C₁-C₆ alcohols, such as ethanol, isopropanol, propanol or butanol; or glycols, such as glycerol, propylene glycol, 1,3-butylene glycol, dipropylene glycol or polyethylene glycols comprising from 4 to 16 ethylene oxide units and preferably from 8 to 12.

The composition according to the invention can comprise an oil chosen for example from:
oils of animal or vegetable origin formed by esters of fatty acids and of polyols, in particular liquid triglycerides, for example sunflower, maize, soybean, avocado, jojoba, cucumber, grape seed, sesame or hazelnut oils, fish oils, glycerol tricaproate/caprylate, or vegetable or animal oils of formula R₉COOR₁₀ in which R₉ represents the residue of a higher fatty acid comprising from 7 to 29 carbon atoms and R¹⁰ represents a linear or branched hydrocarbon chain comprising from 3 to 30 carbon atoms, in particular an alkyl or alkenyl chain, for example Purcellin oil or liquid jojoba wax;
natural or synthetic essential oils, such as, for example, oils of eucalyptus, lavandin, lavender, vetiver, litsea cubeba, lemon, santal, rosemary, chamomile, savory, nutmeg, cinnamon, hyssop, caraway, orange, geranium, cade and bergamot;
synthetic oils, such as parleam oil, polyolefins and esters of carboxylic acids which are liquid;
mineral oils, such as hexadecane, isohexadecane and liquid paraffin;
halogenated oils, in particular fluorocarbons, such as fluoroamines, for example perfluorotributylamine, fluorinated hydrocarbons, for example perfluorodecahydronaphthalene, fluoroesters and fluoroethers;
volatile or nonvolatile silicone oils.

The polyolefins which can be used as synthetic oils are preferably poly(α-olefin)s and more particularly those of hydrogenated or nonhydrogenated polybutene type, preferably hydrogenated or nonhydrogenated polyisobutene.

The esters of carboxylic acids which are liquid which can be used as synthetic oils include esters of mono-, di-, tri- or tetracarboxylic acids. The total carbon number of the esters is generally greater than or equal to 10 and preferably less than 100 and more particularly less than 80. These are in particular monoesters of saturated or unsaturated and linear or branched C₁-C₂₆ aliphatic acids and of saturated or unsaturated and linear or branched C₁-C₂₆ aliphatic alcohols, the total carbon number of esters generally being greater than or equal to 10. Use may also be made of esters of C₄-C₂₂ di- or tricarboxylic acids and of C₁-C₂₂ alcohols and esters of mono-, di- or tricarboxylic acids and of di-, tri-, tetra- or pentahydroxylated alcohols comprising 2 to 26 carbon atoms.

Preference is given, among the abovementioned esters, to the use of alkyl palmitates, such as ethyl palmitate, isopropyl palmitate, 2-ethylhexyl palmitate or 2-octyldecyl palmitate; alkyl myristates, such as isopropyl myristate, butyl myristate, cetyl myristate or 2-octyldodecyl myristate; alkyl stearates, such as hexyl stearate, butyl stearate or isobutyl stearate; alkyl malates, such as dioctyl malate; alkyl laurates, such as hexyl laurate and 2-hexyldecyl laurate; isononyl isononanoate; or cetyl octanoate.

The composition according to the invention can be provided in any form including all the formulation forms conventionally used for topical application and in particular in the form of an emulsion (in particular of an oil-in-water or water-in-oil emulsion or of a multiple (W/O/W or O/W/O) emulsion, of an aqueous or oily gel, of an aqueous or oily lotion, of a powder, of a stick, of a spray or of a foam. These compositions are prepared according to the usual methods.

According to a preferred embodiment of the invention, the composition is provided in the form of an emulsion and more particularly of an oil-in-water (O/W) emulsion.

The composition can additionally comprise various adjuvants commonly used in the cosmetics field chosen in particular from emulsifying agents, waxes, fillers, pigments, coloring materials, sequestering agents, fragrances, thickeners, preservatives, cosmetic or dermatological active principles, UV screening agents, moisturizing agents or film-forming polymers.

Of course, a person skilled in the art will take care to choose these adjuvants and/or their amount so that the advantageous properties of the composition according to the invention are not, or not substantially, detrimentally affected by the envisaged addition.

The composition according to the invention can be a composition for caring for or making up the face and/or the body. It is preferably in the form of a leave-in composition. It can thus be a composition for caring for the face and/or body or a makeup composition, such as a product for the complexion (in particular foundation), an eyeshadow, a face powder, an eyeliner, a concealer or product for making up the body.

The composition can also be a hair composition, such as a shampoo, a conditioner, a styling composition or a hair dye.

The invention will now be described with reference to the following examples, given by way of illustration and without implied limitation.

### EXAMPLE 1

PC acrylic polymer microcapsules were prepared according to the following procedure:

A 40% aqueous solution of poly[2-(methacryloyloxy-ethyl)phosphorylcholine] in a water/butanediol mixture (5% of butanediol) sold under the name Lipidure HM by Nippon Oils and Fats was diluted with water to a content of 20% by weight of polymer. 161.8 ml of this aqueous solution were emulsified in 200 ml of dichloromethane comprising 29.4 g of cellulose acetate/propionate (CAP-482-0.5® from Eastman Chemical) in a reactor for 5 minutes using a homogenizer of rotor-stator type at 24 000 revolutions per minute while maintaining the temperature of the mixture below 25° C. 160.6 ml of the primary emulsion thus obtained were introduced into 500 ml of an aqueous solution comprising 1% by weight of poly(vinyl alcohol) (Celvol 203®, Celanese) and comprising 7% of sodium chloride, kept stirred using a Moritz disperser, over 20 minutes at 2000 revolutions per minute, at a temperature of 25° C.

The dichloromethane was subsequently evaporated under vacuum (Buchi B-480 rotary evaporator) at 40° C. After separation by settling and filtration, the microparticles formed were recovered. The microcapsules have a mean diameter of 54 μm.

The degree of encapsulation was measured by quantitative determination of the PC acrylic polymer after mineralization and quantitative determination of the phosphorus using the Biomerieux kit (Lyons, France) “Phosphore UV”.

The degree of encapsulation corresponds to the ratio of the amount of active principle found inside the microcapsules to the total amount of active principle employed during the preparation of the microcapsules.

The microcapsules obtained have a degree of encapsulation of the poly[2-(methacryloyloxyethyl)-phosphorylcholine] of 92% (=17.8/19.6).

### EXAMPLE 2 AND COMPARATIVE EXAMPLE 3

A composition for caring for the skin (Example 2) was prepared which comprises the following ingredients:

| Microcapsules according to Example 1 | 5.6 | g* |
|---|---|---|
| Polyacrylamidomethylpropanesulfonic acid | 0.5 | g |
| partially neutralized with ammonia and crosslinked | | |
| (Hostacerin AMPS from Clariant) | | |
| Preservative | q.s. | |
| Water | q.s. for 100 | g |

*i.e., 1% as active material of polymer comprising a PC group

A composition was prepared which is similar to the above but in which the PC acrylic polymer (Lipidure HM) is not encapsulated but is found directly in the continuous aqueous phase. The composition (Example 3) is as follows:

| | | |
|---|---|---|
| Poly[2-(methacryloyloxyethyl)phosphorylcholine] | 2.5 | g, i.e. 1% AM |
| at 40% in a water/butanediol mixture | | |
| (5% of butanediol) sold under the name | | |
| Lipidure HM by Nippon Oils and Fats | | |
| Water | 96.9 | g |
| Polyacrylamidomethylpropanesulfonic | 0.5 | g |
| acid partially neutralized | | |
| with ammonia and crosslinked | | |
| (Hostacerin AMPS from Clariant) | | |

| | |
|---|---|
| Preservative | q.s. |

The moisturizing properties of the 2 compositions were evaluated on a panel of 25 women selected for having skin on the legs having a high Imperceptible Water Loss (IWL).

The test was carried out by comparing the state of moisturization of a treated area of skin with respect to an untreated area of skin (“bare skin”).

The 2 formulations were applied to the legs twice daily for 2 weeks.

The moisturization was measured by corneometry (measurement of the electrical impedance (EI) of the skin with a Corneometer CM825 SEI-M-0152-COMB-03) at T0 and T15 days.

The ratio (EI treated skin—EI bare skin)/EI bare skin obtained at T15 days was determined for each composition; the following results, expressed as percentage, were obtained:

| | (EI treated skin - EI bare |
|---|---|
| | skin)/EI bare skin |
| Example 2 | +9.2% |
| (invention | |
| Example 3 | +3.9% |
| (outside the | |
| invention) | |

The results obtained show that the composition of Example 2 according to the invention has a better moisturizing property than that of the composition of Example 3. This result confirms that the encapsulation of the PC acrylic polymer makes it possible to improve the moisturizing activity of the polymer.

Furthermore, the panel of women found that the composition according to Example 3 not forming part of the invention is not very moisturizing, tacky and uncomfortable. The encapsulation of the PC acrylic polymer thus makes it possible to improve the properties of the composition comprising such a polymer.

### EXAMPLE 4

PC acrylic polymer and sodium hyaluronate microcapsules were prepared according to the following procedure:

A 40% aqueous solution of poly[2-(methacryloyloxy-ethyl)phosphorylcholine] in a water/butanediol mixture (5% of butanediol) sold under the name Lipidure HM by Nippon Oils and Fats was diluted with water to a content of 10% by weight of polymer and then 0.5% by weight of sodium hyaluronate sold under the name Cristalhyal® by Soliance was added. 160 ml of the aqueous solution thus prepared was emulsified in 200 ml of dichloromethane comprising 20 g of cellulose acetate/propionate (CAP-482-0.5® from Eastman Chemical) in a reactor for 5 minutes using a homogenizer of rotor-stator type at 24 000 revolutions per minute while maintaining the temperature of the mixture below 25° C. 250 ml of the primary emulsion thus obtained were introduced into 500 ml of an aqueous solution comprising 1% by weight of poly(vinyl alcohol) (Celvol 203®, Celanese) and 0.5% by weight of sodium chloride, kept stirred using a Moritz disperser, over 20 minutes at 2000 revolutions per minute, at a temperature of 25° C.

The dichloromethane was subsequently evaporated under vacuum and the microcapsules formed were recovered after separation by settling and filtration. The microcapsules have a mean diameter of 49 μm. The degree of encapsulation of the acrylic polymer comprising a PC group is 70%.

The composition of the microcapsule powder obtained is as follows:

| | | |
|---|---|---|
| Cellulose acetate/propionate | 11.1 | g |
| (CAP-482-0.5 ® from Eastman Chemical) | | |
| Poly[2-(methacryloyloxyethyl)phosphorylcholine] | 22.2 | g AM |
| (Lipidure HM ® from NOF) | | |
| Sodium hyaluronate (Cristalhyal ® | 0.33 | g |
| from Soliance) | | |
| Demineralized water | 66.37 | g |

### EXAMPLE 5

A composition for caring for the skin was prepared in the form of an O/W emulsion comprising the following ingredients:

Phase A

| | | |
|---|---|---|
| PEG-20 methyl glucose sesquistearate | 2 | g |
| (Glucamate SSE 20 from Noveon) | | |
| Disodium salt of EDTA | 0.1 | g |
| Glycerol | 3 | g |
| Preservative | 0.2 | g |
| Demineralized water | 53.5 | g |

Phase B

| | | |
|---|---|---|
| Methyl glucose sesquistearate | 2 | g |
| (Glucate SS from Noveon) | | |
| Mixture of stearyl alcohol and of | 2 | g |
| ceteareth-20 (Ritapro 200 from Rita) | | |
| Cyclohexasiloxane | 6 | g |
| Isocetyl stearate | 8 | g |
| Dicaprylyl carbonate | 3 | g |
| UV screening agents | 4.9 | g |
| Preservative | 0.1 | g |

Phase C

| | | |
|---|---|---|
| Acrylamide/sodium acrylamido-2-methylpropane- | 1 | g |
| sulfonate copolymer as a 40% inverse emulsion | | |
| in isoparrafin/water (Sepigel 305 from Seppic) | | |
| Polyacrylamidomethylpropanesulfonic acid | 1.2 | g |
| partially neutralized with ammonia and crosslinked | | |
| (Hostacerin AMPS from Clariant) | | |

Phase D

| | | |
|---|---|---|
| Aluminum Starch Ocetenylsuccinate | 3 | g |

Phase E

| | | |
|---|---|---|
| Microparticles of Example 4 | 10 | g |

The emulsion is prepared according to known emulsification techniques.

The composition applied to the skin confers good moisturizing and softness properties.

### EXAMPLE 6

O/W emulsion comprising the microcapsules of Example 4

Phase A

| | | |
|---|---|---|
| Demineralized water | 40.69 | g |
| Disodium salt of ethylenediaminetetraacetic | 0.1 | g |
| acid | | |
| Glycerol | 3 | g |
| Preservative | 0.2 | g |

Phase B

| Polyglyceryl-3 methyl glucose distearate | 1.5 | g |
|---|---|---|
| (Tego Care 450 from Goldschmidt) | | |
| Glyceryl stearate | 1.5 | g |
| Stearyl alcohol | 1 | g |
| Myristic acid | 2 | g |
| Apricot oil | 7 | g |
| Isocetyl stearate | 8 | g |
| Preservative | q.s. | |
| Cyclohexasiloxane | 2 | g |
| Dimethiconol | 1 | g |

Phase C

| | | |
|---|---|---|
| Crosslinked polyacrylic acid | 0.4 | g |
| Water | 15 | g |

Phase D

| | | |
|---|---|---|
| Xanthan gum | 0.2 | g |
| Water | 7 | g |

Phase E

| | | |
|---|---|---|
| Triethanolamine | 0.4 | g |
| Water | 1 | g |

Phase F

| | | |
|---|---|---|
| Microparticles of Example 4 | 8 | g |

The emulsion is prepared according to known emulsification techniques.

The composition applied to the skin confers good moisturizing and softness properties.

The above written description of the invention provides a manner and process of making and using it such that any person skilled in this art is enabled to make and use the same, this enablement being provided in particular for the subject matter of the appended claims, which make up a part of the original description and including microcapsules comprising a polymeric and/or waxy envelope encapsulating an aqueous medium comprising an acrylic polymer comprising a phosphorylcholine group.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A microcapsule comprising a polymeric and/or waxy envelope encapsulating an aqueous medium comprising at least one acrylic polymer comprising a phosphorylcholine group.

2. The microcapsule according to claim 1, comprising at least one polymer having an acrylic backbone and comprising pendent groups comprising at least one group of following formula (I): 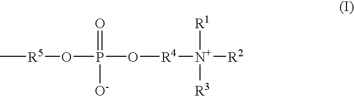
in which R¹, R² and R³ independently denote an alkyl group having from 1 to 8 carbon atoms; R⁴ denotes —(CH₂—CHR₆O)ₘ—(CH₂—CHR₆)ₚ— with R₆ denoting a hydrogen atom or a methyl or ethyl group and m denoting an integer ranging from 0 to 10 and p denoting an integer ranging from 1 to 2; R⁵ denotes —(CH₂)_{g}—, g being an integer ranging from 2 to 10.

3. The microcapsule according to claim 1, comprising at least one polymer obtained by polymerization of an acrylic monomer corresponding to the following formula (II): 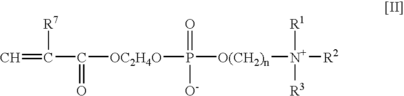
in which R¹, R² and R³ independently denote an alkyl group having from 1 to 8 carbon atoms; n represents an integer ranging from 2 to 4; R⁷ denotes a hydrogen atom or a methyl group.

4. The microcapsule according to claim 3, wherein the acrylic monomer of formula (II) is 2-(meth)acryloyloxyethyl 2′-(trimethylammonio)ethyl phosphate.

5. The microcapsule according to claim 3, wherein the acrylic polymer comprising a phosphorylcholine group is a polymer obtained by polymerization of an acrylic monomer of formula (II) and of one or more additional monomers.

6. The microcapsule according to claim 5, wherein the additional monomer is chosen from methyl (meth)acrylate, ethyl (meth)acrylate, butyl (meth)acrylate, (meth)acrylic acid, (meth)acrylamide, 2-hydroxyethyl (meth)acrylate, ethyl vinyl ether, butyl vinyl ether, N-vinylpyrrolidone, vinyl chloride, ethylene, isobutylene, acrylonitrile, styrene, methylstyrene and (chloromethyl)styrene.

7. The microcapsule according to claim 3, wherein the acrylic polymer comprising a phosphorylcholine group comprises from 40 to 100 mol % of units resulting from the acrylic monomer of formula (II) and from 0 to 60 mol % of units resulting from additional monomer.

8. The microcapsule according to claim 1, wherein the at least one acrylic polymer comprising a phosphorylcholine group is chosen from 2-(methacryloyl-oxyethyl)phosphorylcholine homopolymer, 2-(methacryloyloxyethyl)phosphorylcholine/butyl methacrylate copolymer, 2-(methacryloyloxyethyl)-phosphorylcholine/2-hydroxy-3-methacryloyloxypropyltrimethylammonium chloride copolymer, 2- (methacryloyloxyethyl)phosphorylcholine/butyl methacrylate/sodium methacrylate terpolymer, 2-(methacryloyloxyethyl)-phosphorylcholine/stearyl methacrylate copolymer, and mixtures thereof.

9. The microcapsule according to claim 1, wherein the at lest one acrylic polymer comprising a phosphorylcholine group is chosen from 2-(methacryloyloxyethyl)phosphorylcholine homopolymer,2-(methacryloyloxyethyl)phosphorylcholine/butyl methacrylate copolymer, and mixtures thereof.

10. The microcapsule according to claim 1, comprising 2-(methacryloyloxyethyl)phosphorylcholine homopolymer.

11. The microcapsule according to claim 1, wherein the at least one acrylic polymer comprising a phosphorylcholine group has a weight-average molecular weight ranging from 50 000 to 1 000 000.

12. The microcapsule according to claim 1, wherein the at least one acrylic polymer comprising a phosphorylcholine group is present in the aqueous medium encapsulated in the microcapsules in a content ranging from 0.1% to 40% by weight, with respect to the total weight of the encapsulated aqueous phase.

13. The microcapsule according to claim 1, wherein the envelope comprises a water-insoluble polymer.

14. The microcapsule according to claim 1, wherein the envelope comprises a water-insoluble polymer chosen from:
polycaprolactone;
poly(C₂-C₆ alkylene adipate)s;
polyester polyols of adipic acid and of butanediol;
esters of cellulose and of at least one C₁-C₄ carboxylic acid;
poly(ortho ester)s obtained by polycondensation of polyol, of diol lactide and of 3,9-diethylidene-2,4,8,10-tetraoxaspiro[5.5]undecane;
poly(ethylene glycol)-poly(butylene terephthalate) block polymers;
poly(ethylene glycol terephthalate)/poly(butylene terephthalate) copolymers;
copolymers of styrene and of maleic anhydride, copolymers of styrene and of acrylic acid, styrene-ethylene/butylene-styrene block terpolymers, styrene-ethylene/propylene-styrene block terpolymers and terpolymers of ethylene, of vinyl acetate and of maleic anhydride.

15. The microcapsule according to claim 1, wherein the envelope comprises at least one water-insoluble polymer chosen from cellulose acetate/butyrate and cellulose acetate/propionate.

16. The microcapsule according to claim 1, wherein the envelope comprises at least one wax.

17. The microcapsule according to claim 1, wherein the envelope comprises at least one wax chosen from beeswax, polyglycerolated beeswax, hydrogenated vegetable oils, paraffin wax with a melting point of greater than 45° C. and silicone waxes.

18. The microcapsule according to claim 1, wherein the encapsulated aqueous medium further comprises at least one additional water-soluble polymer.

19. The microcapsule according to claim 18, wherein the at least one additional water-soluble polymer is chosen from poly(vinyl alcohol), polyvinylpyrrolidone, carboxymethylcellulose, poly(carboxylic acid)s and the crosslinked derivatives of these, xanthans, starch, sodium alginate, pectins, chitosan, guar, locust bean, carrageenan and hyaluronic acid.

20. The microcapsule according to claim 1, wherein the encapsulated aqueous medium further comprises at least one polyol which is miscible with water at 25° C. chosen from glycerol, ethylene glycol, propylene glycol, butylene glycol, pentylene glycol and mixtures thereof.

21. The microcapsule according to claim 1, wherein the ratio by weight of the encapsulated aqueous medium to the envelope of the microcapsules is between 0.1/1 and 50/1.

22. The microcapsule according to claim 1, wherein the microcapsule has a mean diameter of between 1 μm and 1000 μm.

23. A process for the manufacture of a microcapsule comprising a polymeric and/or waxy envelope encapsulating an aqueous medium comprising at least one acrylic polymer comprising a phosphorylcholine group, comprising the following successive stages:
(a) dissolution of at least one acrylic polymer comprising a phosphorylcholine group in an aqueous medium to provide an aqueous solution,
(b) emulsification of the aqueous solution obtained in stage (a) in a solution of at least one water-insoluble polymer and/or of at least one wax in a water-immiscible organic solvent to provide a water-in-oil primary emulsion,
(c) emulsification of the water-in-oil primary emulsion obtained in stage (b) in an aqueous solution optionally comprising an agent for stabilizing the emulsion,
(d) removal of the organic solvent by evaporation, giving an aqueous suspension of microcapsules.

24. A composition comprising, in a physiologically acceptable medium, a microcapsule according to claim 1.

25. The composition according to claim 24, further comprising at least one ingredient chosen from solvents which are miscible with water at 25° C., oils, emulsifying agents, waxes, fillers, pigments, coloring materials, sequestering agents, fragrances, thickeners, preservatives, cosmetic or dermatological active principles, UV screening agents, moisturizing agents and film-forming polymers.

26. A method, comprising applying the compositon of claim 24 to a keratinous substance.
